(19) United States
(12) Patent Application Publication
(10) Pub. No.: US 2025/0269202 A1
(43) Pub. Date: Aug. 28, 2025

(54) APPARATUS AND METHOD FOR COMPUTING RADIATION TRANSPORT FOR PROCESSING MONTE CARLO ALGORITHM

(71) Applicant: SEOUL NATIONAL UNIVERSITY R&DB FOUNDATION, Seoul (KR)

(72) Inventors: Sung-Joon YE, Seoul (KR); Chang-Min LEE, Seoul (KR)

(73) Assignee: SEOUL NATIONAL UNIVERSITY R&DB FOUNDATION, Seoul (KR)

(21) Appl. No.: 19/199,638

(22) Filed: May 6, 2025

Related U.S. Application Data
(63) Continuation of application No. PCT/KR2024/001267, filed on Jan. 26, 2024.

(30) Foreign Application Priority Data
Feb. 7, 2023 (KR) 10-2023-0016114

(51) Int. Cl. A61N 5/10

(52) U.S. Cl. CPC A61N 5/1031; A61N 2005/1034

(57) ABSTRACT

According to the present disclosure, a GPU-based radiation transport computing apparatus includes: an interface module configured to communicate with an external device; a memory configured to store a radiation transport computation program; at least one GPU; and a CPU configured to control the execution of the radiation transport computation program. Herein, the radiation transport computation program allocates, in the GPU, a first buffer for processing source particles, a second buffer for processing information about a transport process of radioactive particles, and a third buffer for processing information about various particles generated in a reaction during radiation exposure, selects a buffer to process a thread block that performs a Monte Carlo algorithm based on occupancy rates of the first to third buffers, and performs sampling on the selected buffer through each thread block according to the Monte Carlo algorithm.

### CROSS-REFERENCE TO RELATED APPLICATION

Pursuant to 35 USC 120 and 365(c), this application is a continuation of International Application No. PCT/KR2024/001267 filed on Jan. 26, 2024, and claims the benefit under 35 USC 119(a) of Korean Application No. 10-2023-0016114 filed on Feb. 7, 2023, in the Korean Intellectual Property Office, the entire disclosure of which is incorporated herein by reference for all purposes.

### TECHNICAL FIELD

The technical field of the present disclosure relates to a graphics processing unit (GPU)-based apparatus and method for computing radiation transport for processing a Monte Carlo algorithm.

### BACKGROUND

Radiation therapy is widely used as a treatment method for cancer patients. In order to minimize exposure to normal cells during radiation therapy, a process for accurately calculating the radiation dose is necessary. To this end, a radiation treatment planning system (TPS) is used. It is most important to properly plan the radiation absorbed dose to be exposed to a patient during radiation therapy with the TPS. To this end, the radiation dose irradiated to the human body needs to be calculated accurately and quickly. A Monte Carlo algorithm is the most accurate gold standard method for calculating the radiation dose, but it has the disadvantage of requiring a lot of calculation cost (in terms of time, equipment, etc.). Therefore, most treatment planning systems use an approximate approach through fast convolution or convolution-superposition, which has a relatively fast calculation speed. However, in the case of neutron dose calculation, the reaction patterns are different compared to X-rays and various types of radiation are generated, and, thus, the dose composition becomes complex. Therefore, unlike conventional X-ray and electron beam therapies, a Monte Carlo method is primarily used.

The Monte Carlo algorithm simulates the movement of radioactive particles during radiation exposure by estimating various conditions or reaction parameters and computes all doses transmitted by radiation. Particularly, a Monte Carlo radiation N-Particle transport code (e.g., MCNP, Geant4, PHITS, etc.) is commonly used to simulate particle transport in a voxel model of a target to be irradiated. Herein, the voxel model of the target can be generated from DICOM (Digital Imaging and Communications in Medicine)-based digital medical images, such as CT, MRI, or PET. The Monte Carlo radiation N-Particle transport code is a program that simulates a transport process of one or more particles, such as neutrons, photons, or electrons, and employs probabilistic methodologies to simulate the reactions between radioactive particles and medium particles. The Monte Carlo method is a simulation method for calculating the value of a desired function using randomly extracted random numbers. Since the simulation is performed by generating random numbers for countless radioactive particles, it is challenging to compute the dose using the conventional Monte Carlo algorithm in real time or within a required clinical timeframe.

Particularly, the Monte Carlo algorithm has been executed based on CPU, which takes a lot of time to obtain sufficient statistical uncertainty. Even when using parallel processing servers, a CT-based structure used in the medical field takes several hours to more than a day, making it impractical for clinical use. More specifically, the conventional CPU-based Monte Carlo algorithm processes all tasks sequentially, and, thus, individual threads may simulate different particles or physical phenomena.

Meanwhile, with the recent advent of General-Purpose Graphics Processing Unit (GPGPU) technology, graphics processor acceleration has been applied in various simulation fields. However, it is very difficult to apply the GPGPU technology due to the inherent randomness of the Monte Carlo algorithm. This is because the GPGPU technology shows a sharp performance degradation due to divergent branches occurring when computations differ between threads. Particularly, when the CPU-based Monte Carlo algorithm is directly applied to the GPU, divergent branches occur. Divergent branching will be described in more detail as follows. The minimum unit of threads processed in parallel in a GPU multiprocessor produced by a specific company is called warp, which typically consists of 32 threads. The threads in each warp can execute only one instruction at a time. If the threads in each warp perform different computations due to a code structure, such as an if/else branch, the instructions after the point where a branch occurs are executed sequentially rather than in parallel. In this case, threads that are located in different branches and are not the target of instruction execution are disabled, and, thus, the full performance of the hardware cannot be exhibited. This phenomenon is called divergent branching. In the worst case, all 32 threads execute different instructions. In this case, the computation efficiency drops to 3% compared to the ideal situation. Therefore, to utilize the GPU efficiently, it is necessary to minimize the occurrence of branches within the algorithm.

Accordingly, the present disclosure proposes a GPU-based apparatus and method for computing radiation transport that can solve the above-described problems.

### DISCLOSURE OF THE INVENTION

### Problems to be Solved by the Invention

In view of the foregoing, the present disclosure is conceived to provide an apparatus and method for computing radiation transport based on a Monte Carlo algorithm which can be used on a GPU computing environment.

The problems to be solved by the present disclosure are not limited to the above-described problems. There may be other problems to be solved by the present disclosure.

### Means for Solving the Problems

A first aspect of the present disclosure provides a GPU-based radiation transport computing apparatus, including: an interface module configured to communicate with an external device; a memory configured to store a radiation transport computation program; at least one GPU; and a CPU configured to control the execution of the radiation transport computation program. Herein, the radiation transport computation program allocates, in the GPU, a first buffer for processing source particles, a second buffer for processing information about a transport process of radioactive particles, and a third buffer for processing information about various particles generated in a reaction during radiation exposure, selects a buffer to process a thread block that performs a Monte Carlo algorithm based on occupancy rates of the first to third buffers, and performs sampling on the selected buffer through each thread block according to the Monte Carlo algorithm.

A second aspect of the present disclosure provides a GPU-based radiation transport computing method, including: (a) a process of allocating, in the GPU, a first buffer for processing source particles, a second buffer for processing information about a transport process of radioactive particles, and a third buffer for processing information about various particles generated in a reaction during radiation exposure; (b) a process of selecting a buffer to process a thread block that performs a Monte Carlo algorithm based on occupancy rates of the first to third buffers; and (c) a process of performing sampling on the selected buffer through each thread block according to the Monte Carlo algorithm.

### Effects of the Invention

According to an embodiment of the present disclosure, it is possible to provide a computing system that provides a computation speed at least 100 times faster than a conventional CPU-based Monte Carlo algorithm and can simultaneously simulate various types of radiation, such as neutrons, photons, electrons, positrons, protons, and heavy ions. Also, the present computing system supports existing CT-based DICOM input/output standards, and, thus, it enables dose calculation simulations for advanced radiation therapies, such as conventional X-ray and electron beam therapies or heavy ion and neutron therapies.

Further, the algorithm of the present disclosure can be applied in a multi-GPU environment due to its characteristics and enables computations on a GPU server. A resultant remarkable increase in computation speed enables a real-time response to a patient's treatment plan in a hospital environment. Thus, it is possible to provide an accurate and fast patient dose distribution through Monte Carlo computation.

### BRIEF DESCRIPTION OF THE DRAWINGS

FIG. 1 is a block diagram illustrating a configuration of a radiation transport computing apparatus according to an embodiment of the present disclosure.

FIG. 2A, FIG. 2B, FIG. 2C, and FIG. 2D are diagrams illustrating a buffer reservation process according to an embodiment of the present disclosure.

FIG. 3, FIG. 4, FIG. 5, and FIG. 6 are flowcharts showing a radiation transport computing method according to an embodiment of the present disclosure.

FIG. 7, FIG. 8, FIG. 9, and FIG. 10 are provided to explain a hierarchical dose calculation process according to an embodiment of the present disclosure.

### BEST MODE FOR CARRYING OUT THE INVENTION

Hereafter, embodiments will be described in detail with reference to the accompanying drawings so that the present disclosure may be readily implemented by a person with ordinary skill in the art. However, it is to be noted that the present disclosure is not limited to the embodiments but can be embodied in various other ways. In the drawings, parts irrelevant to the description are omitted for the simplicity of explanation, and like reference numerals denote like parts throughout the whole document.

Throughout this document, the term “connected to” may be used to designate a connection or coupling of one element to another element and includes both an element being “directly connected to” another element and an element being “electronically connected to” another element via another element.

Throughout the whole document, the term “on” that is used to designate a position of one element with respect to another element includes both a case that the one element is adjacent to the other element and a case that any other element exists between these two elements.

Hereinafter, embodiments of the present disclosure will be described in detail with reference to the accompanying drawings.

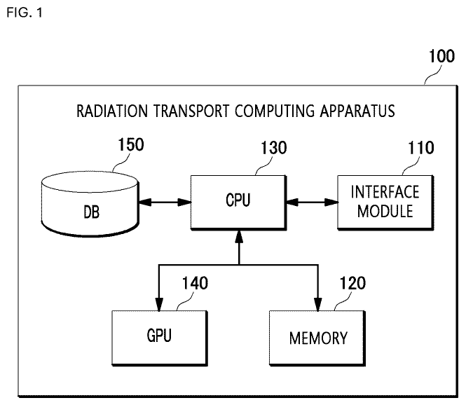

FIG. 1 is a block diagram illustrating a configuration of a radiation transport computing apparatus according to an embodiment of the present disclosure.

A radiation transport computing apparatus 100 includes an interface module 110 configured to exchange data with an external computing device or radiation equipment, a memory 120, a CPU 130, a GPU 140, and a database (DB) 150.

The radiation transport computing apparatus 100 checks information about phase space through a radiation transport simulation for various types of radiation and calculates various radiation data, such as radiation flux, radiation charge, and radiation dose. The radiation transport computing apparatus 100 can pre-calculate a radiation treatment plan prior to operating the radiation equipment. To this end, the radiation transport computing apparatus 100 collects digital images from the external computing device or radiation equipment and creates a voxel model of a target based on the collected digital images. Further, the radiation transport computing apparatus 100 executes a radiation transport computation program to process random branches, which occur in a transport process of radioactive particles, in parallel through the GPU 140, and, thus, it is possible to solve the occurrence of divergent branches in a CPU-based algorithm. Meanwhile, the radiation transport computing apparatus 100 may be implemented with computers or portable devices which can access a network. Herein, the computers may include, for example, a notebook, a desktop, and a laptop. The portable devices are, for example, wireless communication devices that ensure portability and mobility and may include all kinds of handheld-based wireless communication devices such as various smart phones, tablet PCs, and smart watches. Also, the radiation transport computing apparatus 100 may function as a server to supply generated radiation dose data. Herein, the server may operate in a cloud computing service model, such as software as a service (SaaS), platform as a service (PaaS), or infrastructure as a service (IaaS), and may be built in the form of a private cloud, a public cloud, or a hybrid cloud.

The radiation transport computation program is stored in the memory 120. The radiation transport computation program is initially executed by the CPU 130, and the main logic of the radiation transport computation program, such as allocation of a buffer to the GPU or sampling in each buffer, is executed in a state where the radiation transport computation program is loaded on the GPU 140. The radiation transport computation program allocates, in the GPU 140, a first buffer for processing source particles, a second buffer for processing information about a transport process of radioactive particles, and a third buffer for processing information about various particles generated in a reaction during radiation exposure, selects a buffer to process a thread block that performs a Monte Carlo algorithm based on occupancy rates of the first to third buffers, and performs sampling on the selected buffer through each thread block according to the Monte Carlo algorithm.

Meanwhile, the memory 120 is a general name for non-volatile storage devices that can preserve information stored therein without electric power and volatile storage devices that need electric power to preserve information stored therein. The memory 120 may include a magnetic storage medium or a flash storage medium in addition to the volatile storage device requiring power to preserve information stored therein, but the present disclosure is not limited thereto.

Further, the CPU 130 controls general computing operations of the radiation transport computing apparatus 100. For example, the CPU 130 can execute the operating system or manage data received from the external device through the interface module 110. Furthermore, according to an execution request from a manager, the CPU 130 may start the execution of the radiation transport computation program stored in the memory 120, enable the main logic of the radiation transport computation program to be executed on the GPU 140, receive output information produced by the radiation transport computation program on the GPU 140, and transmit the output information to the external device through the interface module 110.

Furthermore, the GPU 140 is a Graphics Processing Unit, which includes General-Purpose computing on Graphics Processing Units (GPGPU). A thread is the minimum unit of an execution flow executed on a CPU or GPU. Particularly, a plurality of threads is processed in parallel within the GPU 130. A plurality of threads (for example, 32 threads) is grouped into a block, which is defined as “thread block” in the present disclosure. When executing a Monte Carlo algorithm, a series of processes of generating a random number for each particle to simulate the state of one particle and processing a result based on the random number may be processed by one thread. The process of each thread includes sampling information about the state of the particle. In this case, the number of threads is optimized based on the specifications of the GPU. Threads are created in consideration of the number of radioactive particles. For example, if the GPU can process up to 10∝threads and simulates 10∂primary particles, source sampling is invoked repeatedly 1,000 times whenever the occupancy rate of a source buffer decreases. Thus, a total of 10∂particles are sampled.

The radiation transport computation program checks the occupancy rate of each buffer in the unit of thread block in which a plurality of threads is grouped and selects a buffer to perform tasks in the thread block. In the present disclosure, at least three buffers are formed on the memory embedded in the GPU 130, and each buffer performs tasks in the thread block.

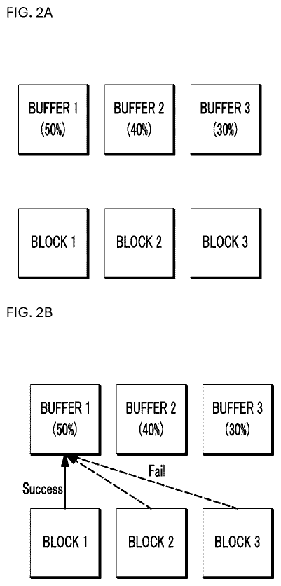

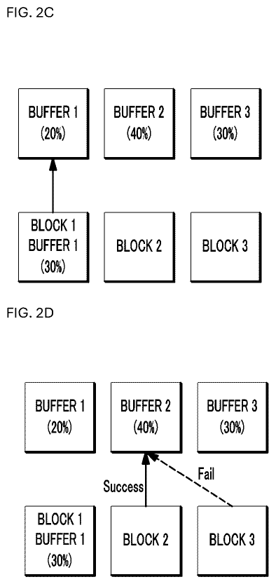

FIG. 2A to FIG. 2D are diagrams illustrating a buffer reservation process according to an embodiment of the present disclosure.

The first to third buffers are allocated in the GPU 130. The first buffer serves as a source buffer for processing source particles, the second buffer serves as a transport buffer for processing information about a transport process of radioactive particles, and the third buffer serves as a reaction buffer for processing information about various particles generated in a reaction during radiation exposure.

The radiation transport computation program selects a buffer to be reserved by a thread block by checking the occupancy rate of each buffer and prioritizing the buffer with the highest occupancy rate. This is because tasks stored in the buffer with the highest occupancy rate need to be processed first to avoid overflow of the buffer. A plurality of thread blocks selects the buffer with the highest occupancy rate from among a plurality of buffers. To avoid the occurrence of a race condition in which different thread blocks select the same buffer at the same time, i.e., to ensure that only one thread block selects a buffer, an atomicCAS operation or the like may be used.

For example, as shown in FIG. 2A and FIG. 2B, if the occupancy rates of the first buffer, the second buffer, and the third buffer are 50%, 40%, and 30%, respectively, each thread block selects the first buffer with the highest occupancy rate and attempts to reserve the first buffer. Only one of the plurality of thread blocks can use the first buffer. At this point, a thread block may be randomly selected from the plurality of thread blocks to use the first buffer, or the thread block that transmits a reservation request first can use the first buffer, or the thread block that will use the first buffer may be determined by some other selection method.

As described above, the thread block that has reserved the first buffer performs sampling based on information stored in the first buffer. As shown in FIG. 2C, when the thread block processes 30% of the information stored in the first buffer, the occupancy rate of the first buffer decreases accordingly. Meanwhile, as shown in FIG. 2D, the other thread blocks attempt to reserve the remaining buffers, checks the occupancy rates of the remaining buffers, and selects and reserves one buffer with the highest occupancy rate from among the remaining buffers.

Referring back to FIG. 1, the DB 150 stores data necessary for the execution of the radiation transport computation program under the control of the CPU 130 or the GPU 140. The DB 150 may be included as a separate component from the memory 120 or built into a part of the memory 120. Further, the DB 150 may store various types of digital medical images or voxel model data transmitted from external device.

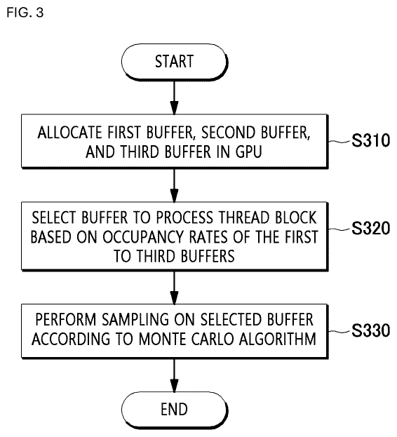

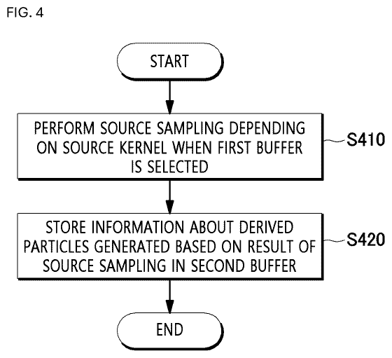

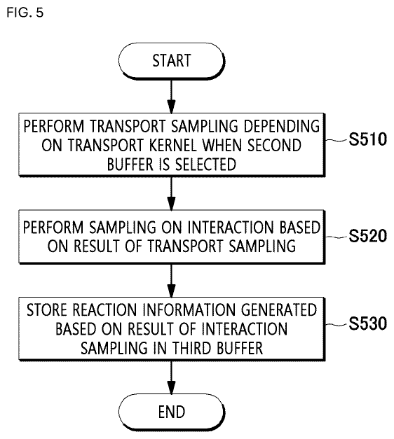

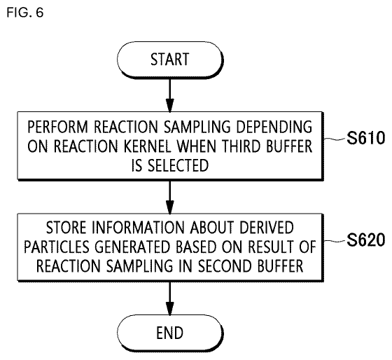

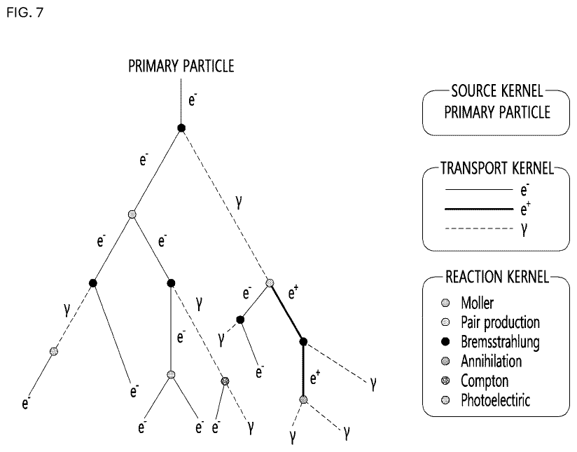

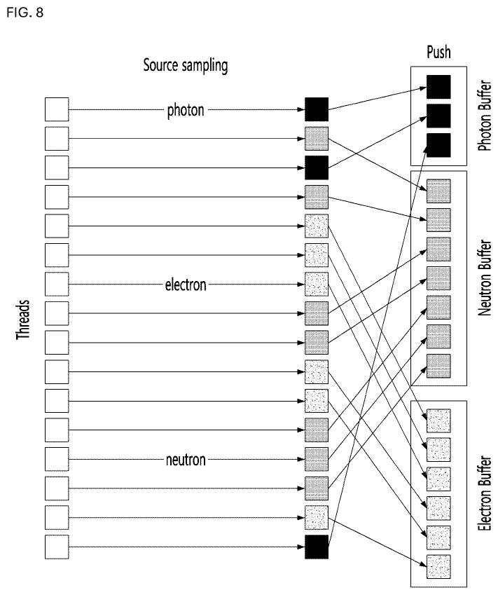

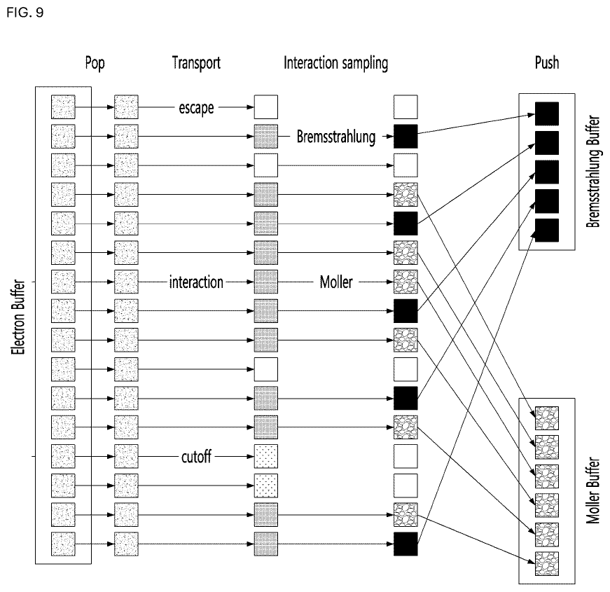

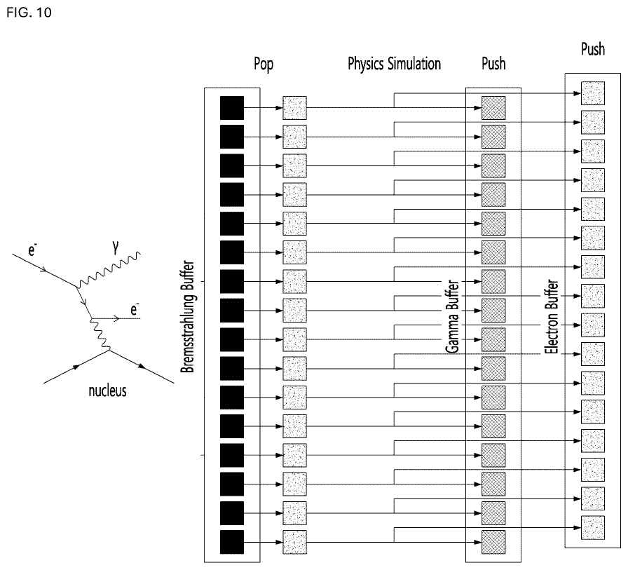

FIG. 3 to FIG. 6 are flowcharts showing a radiation transport computing method according to an embodiment of the present disclosure, and FIG. 7 to FIG. 10 are provided to explain a hierarchical dose calculation process according to an embodiment of the present disclosure.

Referring to FIG. 3, after the radiation transport computation program is executed by a CPU, it is executed on a GPU. In a memory within the GPU, a first buffer for processing source particles, a second buffer for processing information about a transport process of radioactive particles, and a third buffer for processing information about various particles generated in a reaction during radiation exposure are allocated (S310).

Then, a buffer to process a thread block that performs a Monte Carlo algorithm is selected based on the occupancy rates of the first to third buffers (S320).

As described above, as the Monte Carlo algorithm is executed, a large number of threads are generated, and a thread block in which the thread blocks are grouped selects a specific buffer to perform sampling. In this case, the thread block selects the buffer with the highest occupancy rate.

Then, the thread block performs sampling on the buffer selected in the process S320 through each thread block according to the Monte Carlo algorithm (S330).

An operation of a source kernel shown in FIG. 4, an operation of a transport kernel shown in FIG. 5, and an operation of a reaction kernel shown in FIG. 6 are executed depending on the type of the selected buffer.

Referring to the operation of the source kernel shown in FIG. 4, when the first buffer is selected, source sampling is performed on individual particles stored in the first buffer according to the Monte Carlo algorithm depending on the source kernel (S410) and information about derived particles generated based on a result of the source sampling is stored in the second buffer (S420).

As shown in FIG. 7, primary particles (photons, electrons, protons, neutrons, etc.) included in radiation collide or react with other atoms to generate new particles during transport, and branches occur in this process. In the present disclosure, information about the primary particles is managed in the first buffer, information about the transport process of various particles is managed in the second buffer, and the information about derived particles generated in a reaction is managed in the third buffer. As described above, all information required for computation of radiation transport is divided into three layers and managed, and each layer is separated and processed in parallel. Thus, it is possible to minimize the occurrence of divergent branches in the CPU.

Accordingly, in the operation of FIG. 4, information corresponding in number to the primary particles is first stored in the first buffer. Then, when a thread block selects the first buffer, the thread block performs source sampling until all the primary particles stored in the first buffer are processed. To this end, the thread block invokes a SourceSampling( ) function of the Monte Carlo algorithm, performs sampling of the position and energy of individual particles. Information about the sampled particles is stored in a phase-space data structure and then transferred to the second buffer. In this case, a mixed source of various types of particles is used during source sampling. Thus, if transport sampling of the particles is performed after the source sampling, divergent branches may occur depending on the type of each particle. Therefore, the transport sampling is not performed during the source sampling.

FIG. 8 illustrates an example of a detailed operation of the source kernel. However, in FIG. 8, the type of particle is not initially defined for each thread. As each thread is executed, the type of particle is determined by source sampling and information about each particle is stored in the second buffer. At this point, in the second buffer, sub-buffers (electron buffer, neutron buffer, and photon buffer) are generated for respective particle types to separately store the information about each particle.

In the operation of FIG. 5, when a thread block selects the second buffer, it performs transport sampling on particles stored in the second buffer according to the Monte Carlo algorithm depending on the transport kernel (S510), performs sampling on an interaction of the Monte Carlo algorithm based on a result of the transport sampling (S520), and stores reaction information generated based on a result of the interaction sampling in the third buffer (S530). To this end, the thread block invokes a ParticleTranport( ) function of the Monte Carlo algorithm, and when interactions occur as a result of sampling, the thread block stores the information in a phase-space data structure and then transfers it to the third buffer.

FIG. 9 illustrates an example of a detailed operation of the transport kernel. In the second buffer, sub-buffers may be generated for respective particle types (electrons, protons, positrons, neutrons, photons, heavy ions, etc.). Further, threads are generated to perform transport for respective particle types. For example, particles may escape, interact, or undergo cutoff, and some of these particles may be terminated during transport without storing data in the buffer. For particles that have been transported normally, interactions are sampled and various reactions may occur during the sampling. For example, various reactions, such as Bremsstrahlung or Moller scattering, may occur.

The reaction information generated based on a result of the interaction sampling is stored in the third buffer.

In the operation of FIG. 6, when a thread block selects the third buffer, it performs reaction sampling on the reaction information stored in the third buffer according to the Monte Carlo algorithm depending on the reaction kernel (S610), and stores information about derived particles generated based on a result of the reaction sampling in the second buffer (S620). To this end, the thread block invokes a ReactionSampling( ) function of the Monte Carlo algorithm. When derived particles are generated as a result of sampling, the information is transferred to the second buffer, repeating the transport process, stores the information about derived particles generated based on a result of sampling in a phase-space data structure and then transfers it to the second buffer.

FIG. 10 illustrates an example of a detailed operation of the reaction kernel. In the third buffer, various types of reaction information is stored, and sub-buffers may be generated for respective types of physical reactions, such as Rayleigh scattering, Compton scattering, Bremsstrahlung, pair annihilation, pair production, Moller scattering, Bhabha scattering, and inelastic scattering. Since transport is not performed, there is no case where a specific thread is terminated. For example, in the case of Bremsstrahlung shown in FIG. 10, the particles generated are one photon and one electron, and derived particles generated based on a reaction sampling result are stored in a corresponding particle buffer in the second buffer depending on the type of particle.

As described above, each thread block selects a buffer with the highest occupancy rate based on the occupancy rates of the first to third buffers and processes information stored in each buffer. While the thread block processes information stored in the first buffer, information about particles is newly sampled and then stored in the second buffer. Therefore, the process of processing the information stored in the first buffer results in an increase in occupancy rate of the second buffer. Similarly, while the thread block processes information stored in the second buffer, reaction information is newly sampled and then stored in the third buffer. Therefore, the process of processing the information stored in the second buffer results in an increase in occupancy rate of the third buffer. Likewise, while the thread block processes information stored in the third buffer, information about derived particles is newly sampled and then stored in the second buffer. Therefore, the process of processing the information stored in the third buffer results in an increase in occupancy rate of the first buffer. This series of processes can be repeatedly performed until all the primary particles stored in the first buffer are processed.

The radiation transport computing method according to an embodiment of the present disclosure can be embodied in a storage medium including instruction codes executable by a computer such as a program module executed by the computer. A computer-readable medium can be any usable medium which can be accessed by the computer and includes all volatile/non-volatile and removable/non-removable media. Further, the computer-readable medium may include all computer storage media. The computer storage media include all volatile/non-volatile and removable/non-removable media embodied by a certain method or technology for storing information, such as computer-readable instruction code, a data structure, a program module or other data.

The method and system of the present disclosure have been explained in relation to a specific embodiment, but their components or a part or all of their operations can be embodied by using a computer system having general-purpose hardware architecture.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A Graphics Processing Unit (GPU)-based radiation transport computing apparatus, comprising:
an interface module configured to communicate with an external device;
a memory configured to store a radiation transport computation program;
at least one GPU; and
a CPU configured to control the execution of the radiation transport computation program,
wherein the radiation transport computation program allocates, in the GPU, a first buffer for processing source particles, a second buffer for processing information about a transport process of radioactive particles, and a third buffer for processing information about various particles generated in a reaction during radiation exposure, selects a buffer to process a thread block that performs a Monte Carlo algorithm based on occupancy rates of the first to third buffers, and performs sampling on the selected buffer through each thread block according to the Monte Carlo algorithm.

2. The GPU-based radiation transport computing apparatus of claim 1,
wherein when the first buffer is selected, the radiation transport computation program performs source sampling on individual particles stored in the first buffer according to the Monte Carlo algorithm depending on a source kernel, and stores information about a type of particles determined based on a result of the source sampling in the second buffer.

3. The GPU-based radiation transport computing apparatus of claim 1,
wherein when the second buffer is selected, the radiation transport computation program performs transport sampling on particles stored in the second buffer according to the Monte Carlo algorithm depending on a transport kernel, performs sampling on an interaction of the Monte Carlo algorithm based on a result of the transport sampling, and stores reaction information generated based on a result of the interaction sampling in the third buffer.

4. The GPU-based radiation transport computing apparatus of claim 1,
wherein when the third buffer is selected, the radiation transport computation program performs reaction sampling on reaction information stored in the third buffer according to the Monte Carlo algorithm depending on a reaction kernel, and stores information about derived particles generated based on a result of the reaction sampling in the second buffer.

5. The GPU-based radiation transport computing apparatus of claim 1,
wherein the radiation transport computation program enables the thread block to select a buffer with the highest occupancy rate from among the first to third buffers.

6. A Graphics Processing Unit (GPU)-based radiation transport computing method, comprising:
(a) allocating, in the GPU, a first buffer for processing source particles, a second buffer for processing information about a transport process of radioactive particles, and a third buffer for processing information about various particles generated in a reaction during radiation exposure;
(b) selecting a buffer to process a thread block that performs a Monte Carlo algorithm based on occupancy rates of the first to third buffers; and
(c) performing sampling on the selected buffer through each thread block according to the Monte Carlo algorithm.

7. The radiation transport computing method of claim 6,
wherein the process (c) includes:
when the first buffer is selected in the process (b), performing source sampling on individual particles stored in the first buffer according to the Monte Carlo algorithm depending on a source kernel, and storing information about a type of particles determined based on a result of the source sampling in the second buffer;
when the second buffer is selected in the process (b), performing transport sampling on particles stored in the second buffer according to the Monte Carlo algorithm depending on a transport kernel, performing sampling on an interaction of the Monte Carlo algorithm based on a result of the transport sampling, and storing reaction information generated based on a result of the interaction sampling in the third buffer; and
when the third buffer is selected in the process (b), performing reaction sampling on reaction information stored in the third buffer according to the Monte Carlo algorithm depending on a reaction kernel, and storing information about derived particles generated based on a result of the reaction sampling in the second buffer.

8. The radiation transport computing method of claim 6,
wherein in the process (b), the thread block selects a buffer with the highest occupancy rate from among the first to third buffers.

9. A non-transitory computer-readable storage medium that stores a computer program to implement the radiation transport computing method of claim 6.
